(19) United States
(12) Patent Application Publication
(10) Pub. No.: US 2018/0192918 A1
(43) Pub. Date: Jul. 12, 2018

(54) UTILITY GEAR INCLUDING CONFORMAL SENSORS

(71) Applicant: MC10, INC., Lexington, MA (US)

(72) Inventor: Barry Ives, Voorhees, NJ (US)

(21) Appl. No.: 15/869,371

(22) Filed: Jan. 12, 2018

Related U.S. Application Data
(63) Continuation of application No. 14/510,868, filed on Oct. 9, 2014, now abandoned.
(60) Provisional application No. 61/888,946, filed on Oct. 9, 2013.
(60) Provisional application No. 62/058,318, filed on Oct. 1, 2014.

(51) Int. Cl. A61B 5/11; A61B 5/00; A61B 5/0492

(52) U.S. Cl. CPC A61B 5/112; A61B 5/6828; A61B 5/6804; A61B 5/6823; A61B 5/0492

(57) ABSTRACT

A system includes a plurality of conformal sensors and a central controller. Each conformal sensor includes a processing portion and an electrode portion. The electrode portion is configured to substantially conform to a portion of an outer skin surface of a subject and to sense electrical pulses generated by muscle tissue of the subject. The sensed electrical pulses are transmitted from the electrode portion to the processing portion as raw analog signals for onboard processing thereof by the processing portion of the conformal sensor. The processing portion is configured to create digital signals representative of the raw analog signals. The central controller is coupled to each of the plurality of conformal sensors and is configured to receive the digital signals from each of the plurality of conformal sensors.

### CROSS-REFERENCE TO RELATED APPLICATIONS

This application claims the benefit of U.S. Provisional Application Nos. 61/888,946, filed Oct. 9, 2013 (Attorney Docket No. 072044-100042PL01), and 62/058,318, filed Oct. 1, 2014 (Attorney Docket No. 072044-100041PL03), each of which is hereby incorporated by reference herein in its entirety.

### FIELD OF THE INVENTION

The present invention relates generally to conformal sensors and, more particularly, to utility gear including conformal sensors for use in, for example, sending signals and/or data to drive mechanical structures of the utility gear.

### BACKGROUND

Physiological sensing of humans presents an opportunity to manage assistive power to a subject in a manner that mimics decentralized proprioception (the ability to sense the position and location and orientation and movement of the body and its parts). Despite the promise of augmented human proprioception in prior systems, previous efforts at real time physiological sensing in field environments have met with a number of limitations, including motion, contact, and pressure artifacts of sensors, sensitivity to environmental factors such as heat, humidity, rain, etc., as well as power and data routing limitations that render the most robust solutions unwearable, and wearable solutions too intermittent or noisy for real-time use. The present disclosure is directed to solving these and other problems.

### SUMMARY OF THE INVENTION

A system includes a plurality of conformal sensors and a central controller. Each conformal sensor includes a processing portion and an electrode portion. The electrode portion is configured to substantially conform to a portion of an outer skin surface of a subject and to sense a parameter of the subject. The electrode portion generates a parameter signal which is transmitted from the electrode portion to the processing portion. The processing portion is configured to create processed signals based on the parameter signal. The central controller is coupled to each of the plurality of conformal sensors and is configured to receive the processed signals from each of the plurality of conformal sensors.

A system includes a plurality of conformal sensors and a central controller. At least a portion of each of the conformal sensors is configured to substantially conform to a portion of an outer skin surface of a subject and to sense a parameter of the subject and generate a parameter signal based on the sensed parameter. The central controller is coupled to each of the plurality of conformal sensors and is configured to receive the parameter signals from each of the plurality of conformal sensors.

A system includes a plurality of conformal sensors and a central controller. Each conformal sensor includes a processing portion and an electrode portion. The electrode portion is configured to substantially conform to a portion of an outer skin surface of a subject and to sense electrical pulses generated by muscle tissue of the subject. The sensed electrical pulses are transmitted from the electrode portion to the processing portion as raw analog signals for onboard processing thereof by the processing portion of the conformal sensor. The processing portion is configured to create digital signals representative of the raw analog signals. The central controller is coupled to each of the plurality of conformal sensors and is configured to receive the digital signals from each of the plurality of conformal sensors.

A system for monitoring physiological performance of a mammal includes a plurality of conformal sensors and a central controller. Each conformal sensor includes a processing portion and an electrode portion. The electrode portion is configured to substantially conform to a portion of an outer skin surface of the mammal and to sense electrical pulses generated by muscle tissue of the mammal. The sensed electrical pulses are transmitted from the electrode portion to the processing portion as raw analog signals for onboard processing thereof by the processing portion of the conformal sensor. The processing portion is configured to create digital signals representative of the raw analog signals. The central controller is coupled to at least each of the plurality of conformal sensors. The central controller is configurable to (1) receive the digital signals from each of the plurality of conformal sensors; (2) compare the received digital signals with physiological templates stored in a memory device accessible by the central controller to determine a physiological status for the mammal; and (3) based on the determined physiological status, the central controller causing an action to occur within the system.

A system for monitoring physiological performance of a subject includes a plurality of conformal sensors and a central processing unit. Each conformal sensor includes an electrode for monitoring muscle tissue activity of the subject by measuring analog electrical signals output by the muscle tissue that are indicative of muscle tissue movement. The analog signal is received by a processor chip within each of the plurality of conformal sensors. The processor chip is configured to digitize and filter noise from the analog signal to generate a digital representation of the muscle tissue being monitored. The generated digital representation is stored in at least one first memory. The central processing unit is communicatively coupled with the processor chip of each of the plurality of conformal sensors. The central processing unit includes at least one second memory for storing instructions executable by the central processing unit to cause the central processing unit to: (1) receive the generated digital representations from each of the processor chips of the plurality of conformal sensors; (2) access physiological profiles stored on the at least one second memory or the at least one first memory; and (3) compare the generated digital representations to the physiological profiles to determine a physiological status of the subject.

A system for monitoring physiological performance of a subject includes a physiological conformal sensor and a central controller. The physiological conformal sensor is configured to conform to a portion of an outer skin surface of the subject and to create digital signals representative of physiological data sensed by the physiological sensor. The central controller is coupled to the physiological conformal sensor and is configured to: (1) receive the digital signals from the physiological conformal sensor; (2) determine a physiological stress index based on the received digital signals; and (3) analyze the determined physiological stress index to determine if the subject is at risk or not at risk of reaching dangerous levels of stress.

Additional aspects of the present disclosure will be apparent to those of ordinary skill in the art in view of the detailed description of various implementations, which is made with reference to the drawings, a brief description of which is provided below.

### BRIEF DESCRIPTION OF THE DRAWINGS

FIG. 1A is a perspective view of a utility gear system being worn by a wearer according to some implementations of the present disclosure;

FIG. 1B is a partially exploded perspective view of the utility gear system of FIG. 1A;

FIG. 2A is a front perspective view of the wearer wearing a chest wrap, a pair of thigh wraps, and a pair of calf wraps of the utility gear system of FIG. 1A alongside sample signals sensed by several of the sensors included in the wraps;

FIG. 2B is a back perspective view of the wearer wearing the chest wrap, the pair of thigh wraps, and the pair of calf wraps of the utility gear system of FIG. 1A alongside sample signals sensed by several of the sensors included in the wraps;

FIG. 3 is a perspective view illustrating several of the sensors of the utility gear system of FIG. 1A coupled with a central controller of the utility gear system via a wired connection for supplying power to the sensors and/or for transmitting data therebetween;

FIG. 4A is a front unwrapped view of one of the thigh wraps of the utility gear system of FIG. 1A;

FIG. 4B is a back unwrapped view of the one of the thigh wraps of the utility gear system of FIG. 4A;

FIG. 4C is a perspective view of the one of the thigh wraps of the utility gear system of FIG. 4A shown being wrapped by the wearer to the leg of the wearer according to some implementations of the present disclosure;

FIG. 5A is a pre-filtered sample raw analog signal sensed by a sensor of the utility gear system of FIG. 1A showing muscle activation at a first level of activity;

FIG. 5B is a filtered sample analog signal sensed by a sensor of the utility gear system of FIG. 1A showing muscle activation at the first level of activity with a digitized pulse train signal overlaid thereon;

FIG. 6A is a pre-filtered sample raw analog signal sensed by a sensor of the utility gear system of FIG. 1A showing muscle activation at a second level of activity;

FIG. 6B is a filtered sample analog signal sensed by a sensor of the utility gear system of FIG. 1A showing muscle activation at the second level of activity with a digitized pulse train signal overlaid thereon;

FIG. 7A is a chart used to determine if a wearer of the utility gear of FIG. 1A is at risk or not at risk of reaching dangerous levels of heat and/or exertion stress by looking at data, such as the core body temperature and heart rate of the wearer, according to some implementations of the present disclosure; and

FIG. 7B is a chart used to determine if a wearer of the utility gear of FIG. 1A is at risk or not at risk of reaching dangerous levels of heat and/or exertion stress by looking at a physiological stress index of the wearer, according to some implementations of the present disclosure.

While the present disclosure is susceptible to various modifications and alternative forms, specific implementations have been shown by way of example in the drawings and will be described in detail herein. It should be understood, however, that the present disclosure is not intended to be limited to the particular forms disclosed. Rather, the disclosure is to cover all modifications, equivalents, and alternatives falling within the spirit and scope of the present invention as defined by the appended claims.

### DETAILED DESCRIPTION

While this disclosure is susceptible of implementation in many different forms, there is shown in the drawings and will herein be described in detail preferred implementations of the disclosure with the understanding that the present disclosure is to be considered as an exemplification of the principles of the disclosure and is not intended to limit the broad aspect of the disclosure to the implementations illustrated.

The present disclosure is related to methods, apparatuses, and systems (e.g., utility gear systems) that can analyze data (e.g., physiological data) indicative of body activity such as heart rate, sweat/perspiration rate, temperature, body motion, muscle flexing/movement, etc. for combat performance purposes, activity level monitoring purposes, training purposes, medical diagnosis purposes, medical treatment purposes, physical therapy purposes, clinical purposes, etc.

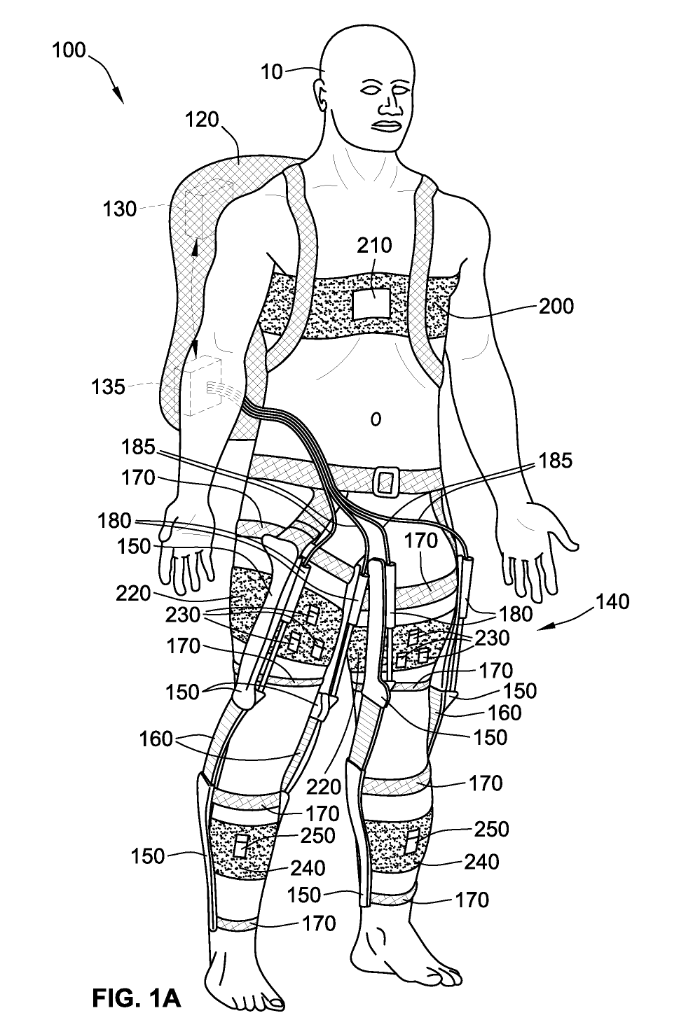

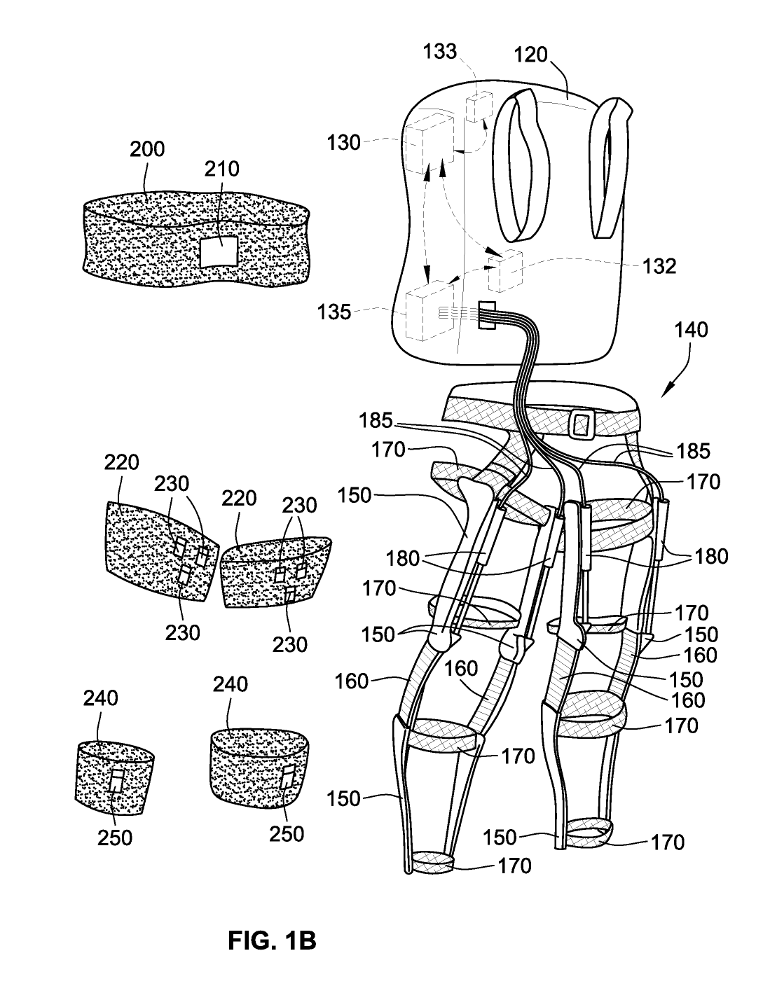

Referring to FIGS. 1A and 1B, a wearer 10 of a utility gear system 100 is shown. The utility gear system 100 includes a storage pack 120 (e.g., back pack), an exoskeleton 140, and a multitude of wraps (e.g., a chest wrap 200, a pair of thigh wraps 220, and a pair of calf wraps 240). Generally, the storage pack 120 includes a central controller 130 that (i) receives data (e.g., processed, filtered digital data/signals) from sensors in the wraps and (ii) uses that data/signals to make decisions on how to control the exoskeleton 140 and/or takes some other type of action like, for example, sending an notification about the wearer's condition/status to a remote location (e.g., a third party like a commanding officer).

The exoskeleton 140 includes many mechanical structures such as a multitude of rigid leg supports 150, bendable knee joint supports 160, flexible straps 170, and hydraulic members 180. The wraps include a chest wrap 200, a pair of thigh wraps 220, and a pair of calf wraps 240. While the utility gear system 100 is shown as including all of these components, more or fewer components can be included in a utility gear system. For example, an alternative utility gear system (not shown) includes the storage pack 120 (e.g., back pack) and a chest wrap 200. For another example, an alternative utility gear system (not shown) includes the storage pack 120 (e.g., back pack), a multitude of rigid leg supports 150, bendable knee joint supports 160, flexible straps 170, hydraulic members 180, a pair of thigh wraps 220, and a pair of calf wraps 240 (i.e., not a chest wrap 200). For another example, an alternative utility gear system (not shown) includes a pair of arm wraps positioned around the wearer's biceps and/or forearms. Thus, various utility gear systems can be formed using the basic components described herein.

As mentioned above, the storage pack 120 includes the central controller 130, which is communicatively coupled with various portions of the utility gear system 100 for controlling operation thereof. In addition to storing the central controller 130, various other components can be stored in the storage pack 120. For example, the storage pack 120 can also store one or more power sources 132 (FIG. 1B) (e.g., battery packs, etc.) for supplying power to the central controller 130 and/or other components of the utility gear system 100, one or more memory devices 133 (FIG. 1B) storing, for example, instructions for operating the central controller 130 according to one or more sets of rules, a hydraulic pump 135 (FIG. 1B), etc. Each of the components in the storage pack 120 can be connected with one or more of the other components via a wired connection and/or a wireless connection. For example, in some implementations, the memory devices 133 are physically wired to the central controller 130, whereas the hydraulic pump 135 is wirelessly controlled by the central controller 130. Yet in some other implementations, all of the components in the storage pack 120 are connected using wired connections to, for example, reduce potential interference issues.

The rigid leg supports 150 are positioned along the lengths of the legs of the wearer 10. Specifically, two of the rigid leg supports 150 are coupled together with one of the bendable knee joint supports 160 to form one half of a leg brace. In the assembled position (FIG. 1A), one leg brace is positioned on both sides of the legs of the wearer 10 and held in place by tightening the flexible straps 170 around the leg of the wearer 10. The flexible straps 170 can be coupled to the leg braces in a variety of manners. For example, the flexible straps 170 can be positioned through slots (not shown) in the rigid leg supports 150. For another example, the flexible straps 170 can be coupled to the rigid leg supports 150 via snap connections, hook and loop fastener connections, glue connections, friction/pressure connections, etc. While not shown, the leg braces can be configured such that a lower end portion of each leg brace contacts the ground surface, an underside of the feet of the wearer 10, a shoe of the wearer 10, or any combination thereof.

Each of the four leg braces also includes one of the hydraulic members 180 coupled thereto. Specifically, in some implementations, the hydraulic members 180 are coupled to the leg braces such that activation of the hydraulic members 180 causes the bendable knee joint supports 160 to bend (not shown), thereby causing/aiding the wearer 10 to move (e.g., walk, run, crawl, etc.). Each of the hydraulic members 180 is coupled to the hydraulic pump 135 in the storage pack 120 by a hydraulic line/tube 185 that supplies the hydraulic member 180 with pressurized hydraulic fluid causing/aiding the above described motion(s). Each of the hydraulic lines 185 is connected to the hydraulic pump 135 in the storage pack 120 which is operable to pump the hydraulic fluid as instructed by the central controller 130 according to, for example, a set of instructions stored in the memory device 133.

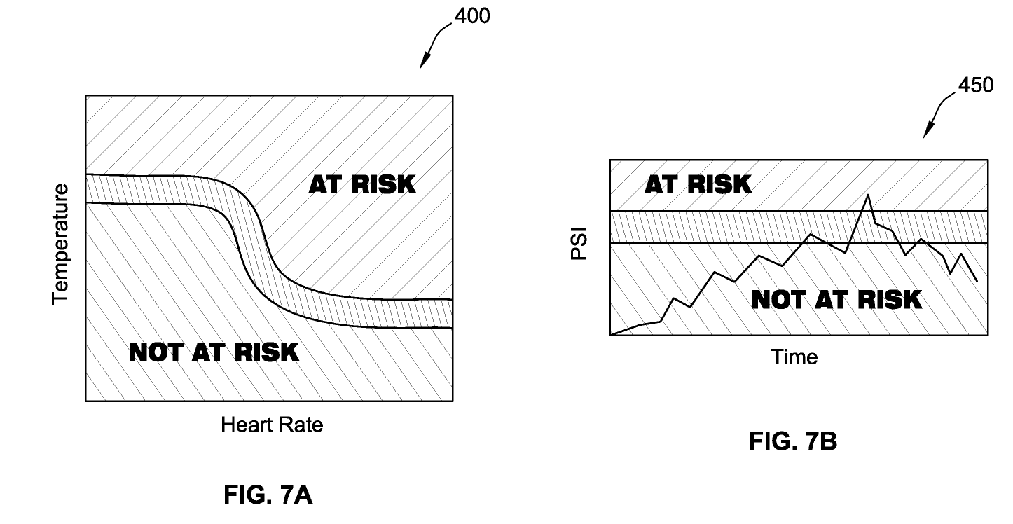

The chest wrap 200 is positioned around the chest or upper torso of the wearer 10 and includes a chest sensor 210 (e.g., a physiological sensor) integrated therein. The chest sensor 210 can be a single sensor or include multiple separate and distinct sensors. For example, the chest sensor 210 can include a heart rate sensor for monitoring a heart rate of the wearer 10 and a core temperature sensor for monitoring/estimating a core body temperature of the wearer 10. In some implementations, the chest sensor 210 is used to determine a physiological stress index (PSI) that can be used, in conjunction with a chart (e.g., charts 400, 450 of FIGS. 7A and 7B), to determine if the wearer 10 is at risk or not at risk of reaching dangerous levels of heat and/or exertion stress by looking at data from the chest sensor 210. Various other sensors can be included in the chest sensor 210, such as, for example, an electromyography (EMG) sensor, a sweat rate/perspiration sensor, a respiration sensor, and an inertial sensor, an accelerometer sensor, an electrocardiogram sensor, an electroencephelogram sensor, etc. The chest sensor 210 is communicatively connected with the central controller 130 to supply data/signals thereto. The connection can be wired and/or wireless.

The thigh wraps 220 are positioned around the thighs of the wearer 10 and include a multitude of sensors 230 integrated therein. By “thigh” it is meant the portion of the leg of wearer 10 between the hips and the knees, which includes the quadriceps muscles (e.g., vastii and rectus femoris) and the hamstring muscles (e.g., biceps femoris and semitendinosus). The sensors 230 are electromyography (EMG) sensors for monitoring electric pulses generated by the muscles of the wearer 10, which indicate muscle movement and/or muscle activity. By positioning the thigh wraps 220 as shown (FIG. 1A), the integrated sensors 230 are automatically positioned adjacent to specific muscles (e.g., quadriceps and hamstrings) in the thighs of the wearer 10. Each of the sensors 230 is communicatively connected with the central controller 130 to supply data/signals thereto. The connection can be wired (shown in FIG. 3) and/or wireless (shown in FIG. 1A). Various other sensors can be included in the thigh wraps 220, such as, for example, temperature sensor, a pulse rate sensor, a sweat rate/perspiration sensor, a respiration sensor, and an inertial sensor, an accelerometer sensor, an electrocardiogram sensor, an electroencephelogram sensor, etc.

Similarly, the calf wraps 240 are positioned around the calves of the wearer 10 and includes a multitude of sensors 250 integrated therein. By “calf” it is meant the portion of the leg of wearer 10 between the knees and the feet, which includes the calf muscles (e.g., gastrocnemius) and the shin muscles (e.g., tibialis anterior). The sensors 250 are electromyography (EMG) sensors for monitoring electric pulses generated by the muscles of the wearer 10, which indicate muscle movement and/or muscle activity. By positioning the calf wraps 240 as shown (FIG. 1A), the integrated sensors 250 are automatically positioned adjacent to specific muscles (e.g., calves and shins) in the lower legs of the wearer 10. Each of the sensors 250 is communicatively connected with the central controller 130 to supply data thereto. The connection can be wired (shown in FIG. 3) and/or wireless (shown in FIG. 1A). Various other sensors can be included in the calf wraps 240, such as, for example, temperature sensor, a pulse rate sensor, a sweat rate/perspiration sensor, a respiration sensor, and an inertial sensor, an accelerometer sensor, an electrocardiogram sensor, an electroencephelogram sensor, etc.

The sensors 210, 230, 250 of the wraps 200, 220, 240 can also be called conformal sensors that are flexible and/or stretchable and/or bendable, and are formed from conformal/bendable processing electronics and/or conformable/bendable electrodes disposed in or on a flexible and/or stretchable substrate. The conformal sensors are positioned in close contact with a surface (such as the skin of the wearer 10) to improve measurement and analysis of physiological information as compared with non-conformal sensors. As best shown in FIG. 3, some of the sensors 230, 250 of the present disclosure include a processing portion 234, 254 and an electrode portion 232, 252. The electrode portion 232, 252 can be formed on, in, or coupled to the same flexible substrate as the electrical circuitry of the processing portions 234, 254 (e.g., a single flexible chip/sensor substrate), as shown in FIG. 3, or can be made separable therefrom (e.g., electrically coupled thereto but comprising two or more separate flexible substrates). Each separate processing electronic component within the conformal sensors 210, 230, 250 can also be referred to an island and/or a chip and can include one or more integrated circuits therein.

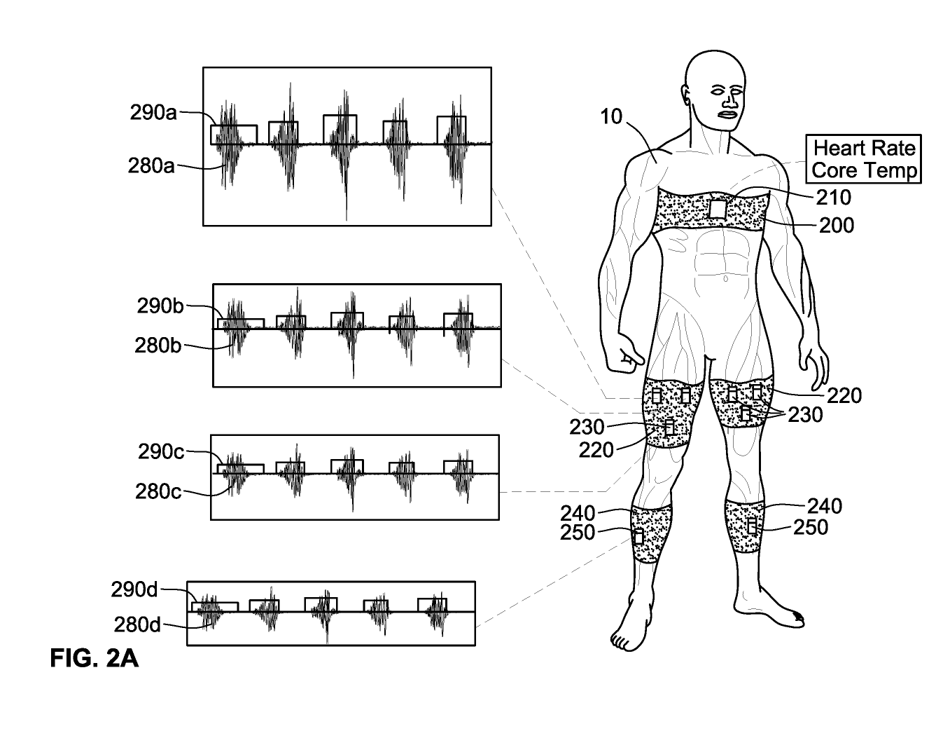

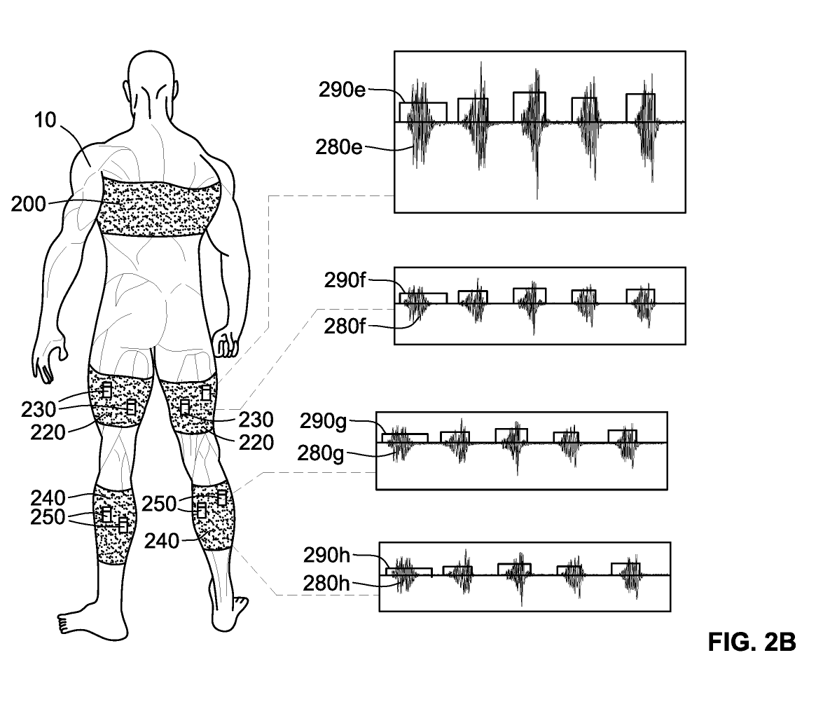

As shown in FIGS. 2A and 2B, in some implementations of the present disclosure, the utility gear system 100 is used to measure the activity of eight different muscle groups in the upper and lower legs of the wearer 10. In some implementations, the electrode portion 232, 252 (FIG. 3) of each of the conformal sensors 230, 250 can include an electromyography (EMG) sensor that is able to collect real-time surface electromyography signals. As represented in the FIGS. 2A and 2B, the analog signals 280a-h collected/read by the EMG sensors 232, 252 can be passed to the processing portion 234, 254 of the conformal sensor 230, 250 to process and/or transmit the collected data via a wired and/or wireless connection. In some implementations, the conformal sensors 230, 250 process the data by filtering noise from the collected data and convert the analog signals 280a-h to digital data such as digital pulse train signals 290a-h that are transmitted to the central controller 130 in the storage pack 120 of the utility gear system 100.

That is, the utility gear system 100 can be configured such that decentralized digital signal processing (DSP) can occur at each conformal sensor 230, 250 at the point of the collection of the data rather than at the central controller 130. Such decentralized digital signal processing results in eliminating off-board analog signal routing, which reduces digital signal bandwidth requirements for the utility gear system 100. Put another way, instead of having to transmit the relatively large analog signals 280a-h from the conformal sensors 210, 230, 250 to the central controller 130, the relatively smaller digital pulse train signals 290a-h can be sent, which requires less power and/or bandwidth allowing for a relatively less expensive system.

The conformal sensors 230, 250 including the EMG sensors 232, 252 are used to evaluate and record electrical activity produced by skeletal muscles. A transducer in each of the EMG sensors 232, 252 detects an electrical potential generated by muscle cells when the muscle cell are electrically or neurologically activated.

Each of the conformal sensors 230, 250 is relatively thin and flexible. For example, in some implementations, the conformal sensors 230, 250 have a thickness of about 500 micrometers to about 5 micrometers such as having a thickness of about 500 micrometers, about 100 micrometers, about 36 micrometers, and/or about 5 micrometers. The thinner the conformal sensors 230, 250, the better the contact the EMG sensors 232, 252 can have with the skin of the wearer 10, which results in relatively fewer motion artifacts in the collected data. For example, a conformal sensor that has a thickness of about 5 micrometers is able to conform to the skin of the wearer 10 with less gaps therebetween as compared with a conformal sensor that has a thickness of about 500 micrometers. Less gaps between the conformal sensor and the skin yields a relatively higher quality/accuracy of the collected data.

Placement of the conformal sensors 230, 250 on the wearer's 10 skin can be made to facilitate analysis of a gait cycle of the wearer 10 and/or to determine fatigue of the wearer 10, performance of the wearer 10, different types of injuries of the wearer 10 (e.g., tendon injury, ligament injury, muscular injury, etc.). Further, placement of the conformal sensors 230, 250 can be made to facilitate a differential comparison of two different muscles, which can enable the utility gear system 100 to determine if the wearer 10 is walking (flat/uphill/downhill), climbing, running (flat/uphill/downhill), crawling, standing for long periods of time, carrying large loads, etc.

The collected data from such specifically placed conformal sensors 230, 250 can be used to determine (e.g., using the central controller 130 and one or more preprogrammed sets of rules) how to intelligently vary the biomechanical assist (e.g., via the exoskeleton 140) to the wearer 10 over a course of exertion/activity of the wearer 10. Such intelligent aid can optimize muscular endurance of the wearer 10, decrease recovery time of the muscles of the wearer 10, and preserve muscular readiness for action of the wearer 10. For example, the central controller 130 and/or some other controller and/or one or more specially programmed processors in communication with the conformal sensors 230, 250 can be used to analyze data measured by the conformal sensors 230, 250 and determine whether the wearer's 10 quadriceps and/or hamstrings are fatigued (e.g., after a long climb, during a walk following the climb, etc.).

In some such implementations, the utility gear system 100 includes a feedback system (not shown) that provides feedback to the wearer 10, such as, for example, instructions to increase tibialis anterior and/or calf activity to allow recovery of the determined fatigued muscle groups (e.g., quadriceps and hamstring muscles). Such feedback can be in the form of an audio track played by a speaker system in the storage pack 120, a video display with a written message built into a helmet or smartphone controlled by the wearer 10, or any other system suitable for communicating such information to the wearer 10. Further, the central controller 130 (or another controller(s) and/or processor(s)) of the utility gear system 100 can continually analyze data from the conformal sensors 230, 250 to determine if the previously determined exhausted muscles have recovered, and in some implementations, provide a follow-up feedback to that effect (e.g., a notification that the wearer's 10 quadriceps and hamstring muscles have recovered and instruct the wearer to balance his/her walking pattern once again).

Referring to FIG. 3, each of the wraps (e.g., the chest wrap 200, the pair of thigh wraps 220, and the pair of calf wraps 240) of the present disclosure can include a multitude of sensors (e.g., 210, 230, 250 as shown). Each of the sensors of the system 100 can be coupled to the central controller 130 via a wired connection, such as, for example, by a micro-USB cable for power and/or digital data transmission. Each of the micro-USB cables that connects a sensor in a specific wrap to the central controller 130 can be routed through a USB hub (not shown) that is integrated with the wrap itself or coupled thereto. In such implementations, the USB hub is then directly connected to the central controller 130 (not the sensors). Such a configuration allows for quick and relatively easy removal of the wrap and associated sensors by physically disconnecting the USB hub from the central controller 130, instead of having to physically disconnect each of the sensors in the wrap (e.g., all five sensors in a thigh wrap 220 do not have to be separately disconnected from the central controller 130, just the micro-USB cable between the USB hub and the central controller 130 is disconnected).

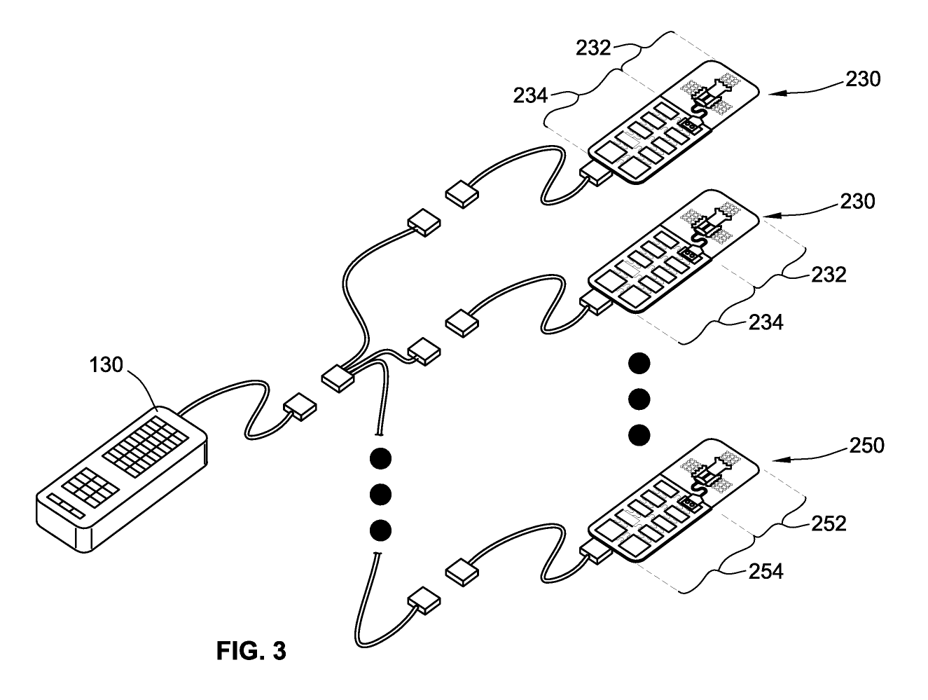

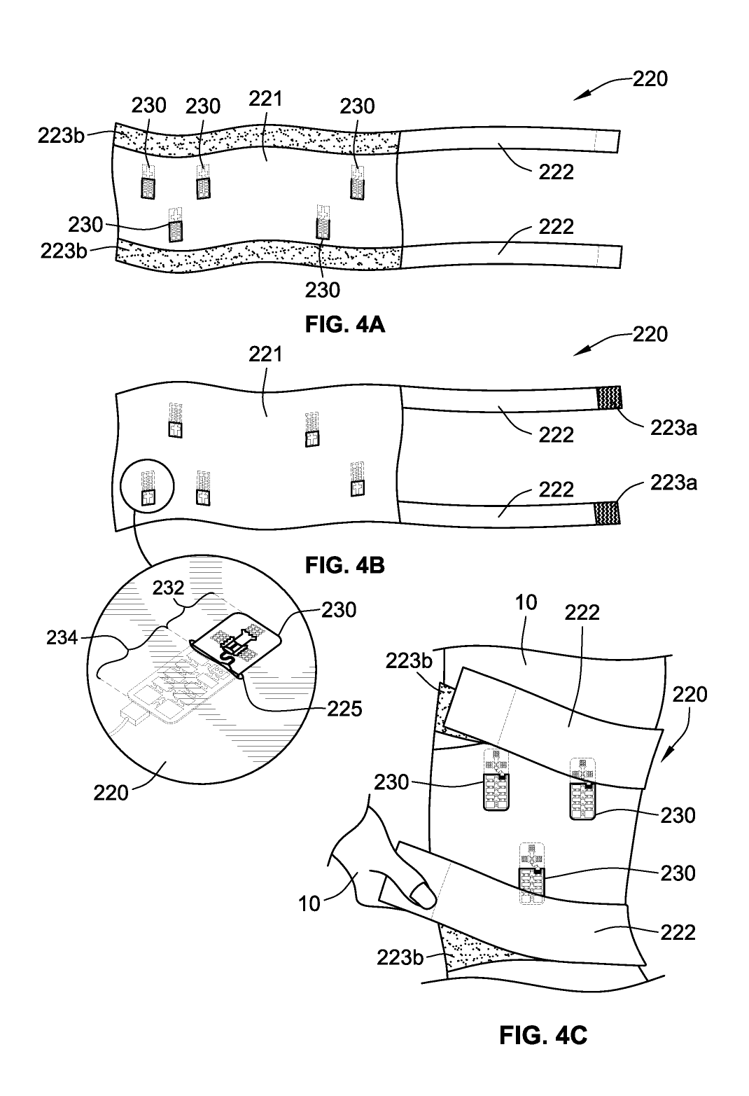

The sensors 210, 230, 250 can be affixed to or coupled with other elements of the utility gear system 100 to facility their use in sensing and processing physiological data. For example, as shown in FIGS. 4A-4C, the conformal sensors 230 of the thigh wrap 220 are embedded in a stretchable fabric portion 221 of the thigh wrap 220 and designed to mate with openings 225 (FIG. 4B) therein for enabling quick attachment and release of the electrode portion 232 of the conformal sensor 230 to/from the skin of the wearer 10. In some implementations, the processing portion 234 of the conformal sensors 230 are positioned in fabric pockets formed in the stretchable fabric portion 221 of the thigh wrap 220 as only the electrode portion 232 needs to contact the skin of the wearer 10. Various additional and/or alternative methods of coupling the conformal sensors 210, 230, 250 to the fabric portions of the wraps 200, 220, 240 are contemplated such that the donning of the wraps 200, 220, 240 automatically positions the conformal sensors 210, 230, 250 therein in the desired location on the skin of the wearer 10.

As best shown in FIG. 4C, to attach the thigh wrap 220 to the leg of the wearer 10, the stretchable fabric portion 221 of the wrap 220 is positioned such that the conformal sensors 230 are positioned adjacent to the desired quadriceps and hamstring muscles. Then the wearer 10 stretches and attaches two straps 222 to the stretchable fabric portion 221 using, for example, hook and loop fasteners 223a,b. As such, the thigh wrap 220 is positioned on the leg of the wearer 10 with the conformal sensors 230 ready to sense muscle activity. If the conformal sensors 230 are wireless sensors, then the donning is complete. However, if the conformal sensors 230 are wired sensors, then one or more wires must be connected from the thigh wrap 220 to the central controller 130 as described above.

Alternative methods of donning the wraps 200 220, 240 are contemplated. For example, the wraps 200, 220, 240 can be slid/pulled onto a limb of the wearer 10 like a stretchable knee brace or the like.

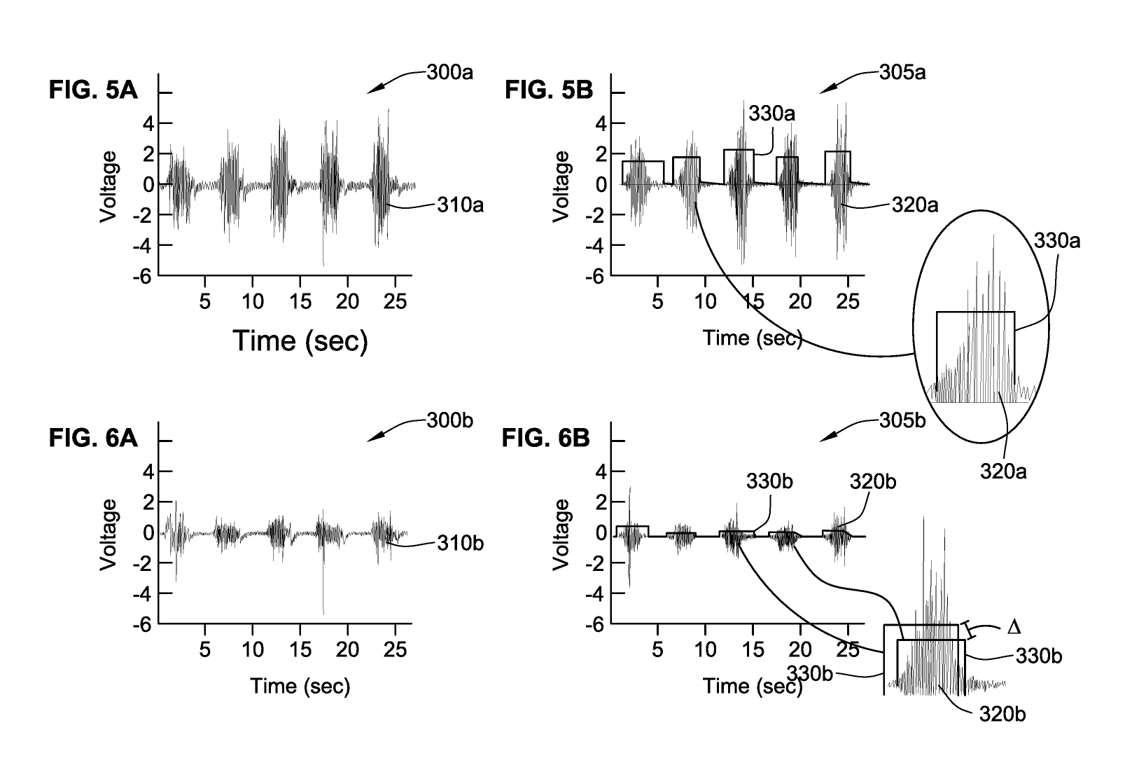

Referring generally to FIGS. 5A-6B, exemplary readings of surface electromyography signals (e.g., voltage) of a muscle of the wearer 10 from one of the conformal sensors 230, 250 are shown. Specifically, the chart 300a of FIG. 5A illustrates a pre-filtered sample raw analog signal 310a sensed by a conformal sensor 230, 250 of the utility gear system 100 showing muscle activation/activity of the wearer 10 at a first level of activity (e.g., lifting a five pound weight). This raw analog signal 310a is transmitted from the electrode portion 232,252 of the conformal sensor 230, 250 to the processing portion 234, 254 of the conformal sensor 230, 250 where the processing portion 234, 254 is designed to filter noise from the raw analog signal 310a, which results in a filtered analog signal 320a as shown in the chart 305a of FIG. 5B. Further, the processing portion 234, 254 is designed to digitize the filtered analog signal by, for example, overlaying a digital pulse train signal 330a on the filtered analog signal 320a which represents the starting, stopping, and amplitude of muscle activity in a digitized format. The digital pulse train signal 330a can also be referred to as a digital signal that is representative of the filtered analog signal 320a.

Similar to FIGS. 5A and 5B, the chart 300b of FIG. 6A illustrates a pre-filtered sample raw analog signal 310b sensed by a conformal sensor 230, 250 of the utility gear system 100 showing muscle activation/activity of the wearer 10 at a second level of activity that is different than the first level of FIGS. 5A and 5B (e.g., lifting a one pound weight). A comparison of the chart 300a of FIG. 5A with the chart 300b of FIG. 6A shows that the amplitude of the raw analog signal 310b is relatively smaller than the raw analog signal 310a, which is due to the muscle being activated by lifting a relatively lighter weight (i.e., one pound vs. five pound). This raw analog signal 310b is transmitted from the electrode portion 232,252 of the conformal sensor 230, 250 to the processing portion 234, 254 of the conformal sensor 230, 250 where the processing portion 234, 254 is designed to filter noise from the raw analog signal 310b, which results in a filtered analog signal 320b as shown in the chart 305b of FIG. 6B. Further, the processing portion 234, 254 is designed to digitize the filtered analog signal 320a by, for example, overlaying a digital pulse train signal 330b on the filtered analog signal 320b which represents the starting, stopping, and amplitude of muscle activity in a digitized format. The digital pulse train signal 330b can also be referred to as a digital signal that is representative of the filtered analog signal 320b.

In some implementations, the processing portion 234, 254 can perform signal processing activities in addition to filtering and digitizing, such as, for example, calculating/extracting statistical information from the analog and/or digitized signals (average amplitude of a set time, peak amplitude, etc.), comparing the analog and/or digital signals from multiple conformal sensors (in some implementations this is done on the central controller 130), etc. As shown in FIG. 6B, a comparison of two bars of the digital pulse train signal 330b are compared (i.e., Delta symbol), which illustrates muscle variability between two different reps of the muscle lifting the same weight. Such knowledge can be used in developing a set of rules to be implemented by the central processor 130 when driving the exoskeleton 140 and/or when analyzing data/signals from the sensors 210, 230, 250 for other purposes.

Generally referring to FIGS. 1A-6B, the conformal sensors 230, 250 can be coupled to controllers and/or processors to analyze data/signals (e.g., surface electromyography signals) from primary muscle groups with good quality, and extract important statistics from the signal for use in development of motor control and power management strategies for the utility gear system 100. In some implementations, the utility gear system 100 including the conformal sensors 210, 230, 250 can be used to facilitate improvement of metabolic efficiency for a healthy test subject under load (e.g., wearer 10). In some implementations, the utility gear system 100 including the conformal sensors 210, 230, 250 can be used to identify markers for fatigue and/or injury at the muscle level, which can influence change of gait strategy implemented by, for example, the central controller 130, and/or an alert the wearer 10 and/or a team leader responsible for the wearer 10 that the wearer 10 may be at risk of reaching a dangerous physiological state/condition.

As described herein, the utility gear system 100 including the conformal sensors 210, 230, 250, can be used to gather physiological data (e.g., surface electromyography signals, skin surface temperature, heart rate, etc.) from the wearer 10. This data can be gathered while the wearer 10 is performing a known, quantifiable, and/or a repeatable exercise, such as, for example, running on a treadmill, walking on a treadmill, crawling, etc., which can be used to develop a baseline profile and/or a physiological template for the wearer 10 under the known/repeatable conditions. This baseline profile and/or a physiological template can be stored (e.g., in the memory device 133) and later used (e.g., by the central processor 130) as a comparison chart with real-time physiological data gathered from the wearer 10 to determine a physiological status/condition of the wearer, such as, for example, if the wearer 10 is exhausted, injured, has a dangerously high heart rate, has a dangerously high core body temperature, performing as expected, performing a specific function (e.g., walking, running, standing, crawling, etc.), etc. Additionally, a database or library of healthy and/or injured baseline profiles/physiological templates, generated from physiological data gathered from the wearer 10 and/or another subject/mammal, can be stored (e.g., in the memory device 133) and used for comparison with real-time physiological data gathered from the wearer 10 to determine if the wearer 10 is exhausted, injured, and/or performing as expected.

For example, to determine if a muscle of interest (e.g., quadriceps) of the wearer 10 is injured, real-time physiological data gathered from the wearer 10 (associated with the muscle of interest) is compared with a library of baseline profiles and/or physiological templates (associated with the muscle of interest of the wearer and/or of another test subject). Specifically, the comparison can include a comparison of raw analog signals, a comparison of filtered analog signals, a comparison of digitized pulse train signals, a comparison of frequencies of the digital pulse train signals, a comparison of amplitudes of the digital pulse train signals, etc. In some implementations, if the amplitude of the digital pulse train signal for one muscle is less than expected for a given activity, that can be an indication of an injury. In some other implementations, if the amplitude of the digital pulse train signal is high and the frequency is low, that can be an indication of an injury. Various other methods for determining injuries using the gathered data are contemplated.

Referring to FIGS. 7A and 7B, charts 400 and 450 are shown for use in determining if the wearer 10 of the utility gear system 100 is at risk or not at risk of reaching dangerous levels of heat and/or exertion stress by looking at data, such as the core body temperature and heart rate of the wearer 10. Specifically referring to FIG. 7A, the chart 400 plots temperature (e.g., core body temperature) of the wearer 10 versus heart rate of the wearer 10. This data can be obtained using the conformal sensor 210 in the chest wrap 200 of the utility gear system 100.

Specifically referring to FIG. 7B, the chart 450 plots a physiological stress index (PSI) determined for the wearer 10 over time. The PSI is an indicator of heat and/or exertion stress of the wearer 10. According to some implementations of the present disclosure, the PSI can be calculated using the following formula:

PSI=5*(T_{core(t)}−T_{core(0)})*(39.5−T_{core(0)})⁻¹+5*(HR₍ₜ₎−HR₍₀₎)*(180−HR₍₀₎)⁻¹

where: T_{core(t)} is the core temperature (Celsius) of the wearer 10 at time t (e.g., ten minutes into an activity); T_{core(0)} is the core temperature (Celsius) of the wearer 10 at time 0 (e.g., zero minutes into the activity); HR₍ₜ₎ is the heart rate (beats per minute) of the wearer 10 at time t (e.g., ten minutes into the activity); and HR₍₀₎ is the heart rate (beats per minute) of the wearer 10 at time 0 (e.g., zero minutes into the activity).

In some implementations, a PSI of seven and a half or greater may be interpreted to be indicative of very high levels of heat/exertion stress. Further, a PSI above seven and a half may be correlated to dangerous levels of heat/exertion stress. In some implementations, the “AT RISK” zone in the chart 400 corresponds to a PSI of seven and a half to ten. In some implementations, if the wearer's 10 PSI is determined to be at or above seven and a half for a predetermined amount of time (e.g., five seconds, two minutes, ten minutes, one hour, etc.), the central controller 130 can be specially programmed to cause the exoskeleton 140 to aid the wearer's 10 physical activity and/or take some other type of action (e.g., send a notice to a commanding officer of the wearer 10, etc.).

As shown and described above, the conformal sensor 210 can include a heart rate sensor and a temperature sensor (e.g., core body temperature sensor), which collectively can be referred to as a PSI monitor as these two conformal sensors together provide the data (e.g., heart rate and core body temperature) used to calculate the PSI. However, it is contemplated that other versions of algorithms and associated methods can be used as a PSI monitor to obtain the same or similar data. For example, an alternative algorithm and associated method can use data indicative of sweat rate and respiration of the wearer 10 to determine the PSI. For another example, an alternative algorithm and associated method can use data indicative of chest skin temperature (opposed to estimated core body temperature) and heart rate of the wearer 10 to determine the PSI.

In some implementations, in addition to the conformal sensors 210, 230, 250 described herein and shown in the drawings, additional sensors can be used with the utility gear system 100 to provide additional data used in evaluating the physiological condition/status of the wearer 10. For example, a wired or wireless sensor can be included in a wrist-borne device (e.g., a watch or bracelet) that senses, for example, ambient temperature, ambient pressure, ambient light, position (e.g., global position, GPS), pulse rate, etc.

In some implementations, a method of assisting the wearer 10 includes monitoring data from the conformal sensors 210, 230, 250, including indications of PSI and/or muscle status (e.g., fatigue, exhaustion, injury) and comparing the monitored data with a baseline profile/physiological template. Based on that comparison and one or more sets of rules, the method determines (1) if the wearer 10 needs assistance by activating an exoskeleton worn by the wearer 10, (2) if a message/alert should be sent to the wearer 10, (3) if a message/alert should be sent to a commanding officer of the wearer 10, etc.

In some implementations, a commanding officer has access to the status of a multitude of warriors (e.g., wearers of separate and distinct utility gear systems). By status it is meant the PSI of the warriors, whether any warrior has an injury, how exhausted each warrior may be based on sensed physiological data, etc. In such implementations, the power in each of the power sources 132 of the utility gear systems 100 being worn by the multitude of warriors can be monitored by the commanding officer and distributed accordingly. For example, the commanding officer might notice that warrior A has full power in her power source 132 and is not exhausted and further that warrior B is low on power in his power source 132 and has an injury. In such an example, the commanding officer can see all of this data on a common display device (e.g., a tablet computer) that is communicatively connected with each active utility gear system 100 and determine that warrior A should give her power source 132 to warrior B for his use.

While the present disclosure has described the utility gear system 100 in reference to a human wearer, the utility gear system 100 or a modified version thereof can be applied to any mammal (e.g., a dog, a horse, etc.).

Alternative Implementations

Alternative Implementation 1. A system comprising: a plurality of conformal sensors, each conformal sensor including a processing portion and an electrode portion, the electrode portion being configured to substantially conform to a portion of an outer skin surface of a subject and to sense electrical pulses generated by muscle tissue of the subject, the sensed electrical pulses being transmitted from the electrode portion to the processing portion as raw analog signals for onboard processing thereof by the processing portion of the conformal sensor, the processing portion being configured to create digital signals representative of the raw analog signals; and a central controller coupled to each of the plurality of conformal sensors and being configured to receive the digital signals from each of the plurality of conformal sensors.

Alternative Implementation 2. The system of Alternative Implementation 1, wherein the central controller is further configured to compare the received digital signals with physiological templates to determine a physiological status of the subject.

Alternative Implementation 3. The system of Alternative Implementation 2, wherein the central controller is further configured to actuate an exoskeleton worn by the subject at various levels of power based on the determined physiological status of the subject.

Alternative Implementation 4. The system of Alternative Implementation 3, wherein the various levels of power include a zero power level, a ten percent power level, a fifty percent power level, a one hundred percent power level, or any other power level in between.

Alternative Implementation 5. A system for monitoring physiological performance of a mammal, the system comprising: a plurality of conformal sensors, each conformal sensor including a processing portion and an electrode portion, the electrode portion being configured to substantially conform to a portion of an outer skin surface of the mammal and to sense electrical pulses generated by muscle tissue of the mammal, the sensed electrical pulses being transmitted from the electrode portion to the processing portion as raw analog signals for onboard processing thereof by the processing portion of the conformal sensor, the processing portion being configured to create digital signals representative of the raw analog signals; and a central controller coupled to at least each of the plurality of conformal sensors, the central controller being configurable to: (i) receive the digital signals from each of the plurality of conformal sensors; (ii) compare the received digital signals with physiological templates stored in a memory device accessible by the central controller to determine a physiological status for the mammal; and (iii) based on the determined physiological status, the central controller causing an action to occur within the system.

Alternative Implementation 6. The system of Alternative Implementation 5, wherein the plurality of conformal sensors are electromyography sensors.

Alternative Implementation 7. The system of Alternative Implementation 5, wherein one or more of the plurality of conformal sensors includes a hard-wired connection to the central controller such that at least some of the electrical signals are received by the central controller via the hard-wired connection.

Alternative Implementation 8. The system of Alternative Implementation 5, wherein one or more of the plurality of conformal sensors are wirelessly connected to the central controller such that at least some of the electrical signals are received by the central controller via the wireless connection.

Alternative Implementation 9. The system of Alternative Implementation 5, wherein one or more of the plurality of conformal sensors are positioned on the outer surface of the mammal adjacent to different muscles.

Alternative Implementation 10. The system of Alternative Implementation 9, wherein the different muscles include the quadriceps muscles, the hamstring muscles, the calf muscles, the biceps muscles, the triceps muscles, or any combination thereof.

Alternative Implementation 11. The system of Alternative Implementation 5, wherein one or more of the plurality of conformal sensors are integral with a stretchable layer of fabric material worn by the mammal such that the conformal sensor device is positioned adjacent to the outer skin surface of the mammal.

Alternative Implementation 12. The system of Alternative Implementation 5, wherein the plurality of conformal sensors are stretchable and bendable.

Alternative Implementation 13. A system for monitoring physiological performance of a subject, the system comprising: a plurality of conformal sensors, each conformal sensor including an electrode for monitoring muscle tissue activity of the subject by measuring analog electrical signals output by the muscle tissue that are indicative of muscle tissue movement, the analog signal being received by a processor chip within each of the plurality of conformal sensors, the processor chip configured to digitize and filter noise from the analog signal to generate a digital representation of the muscle tissue being monitored, the generated digital representation being stored in at least one first memory; and a central processing unit communicatively coupled with the processor chip of each of the plurality of conformal sensors, the central processing unit including at least one second memory for storing instructions executable by the central processing unit to cause the central processing unit to: (a) receive the generated digital representations from each of the processor chips of the plurality of conformal sensors; (b) access physiological profiles stored on the at least one second memory or the at least one first memory; and (c) compare the generated digital representations to the physiological profiles to determine a physiological status of the subject.

Alternative Implementation 14. The system of Alternative Implementation 13, wherein the plurality of conformal sensors includes stretchable processing sensors, each conformal sensor substantially conforming to a portion of an outer surface of the mammal.

Alternative Implementation 15. The system of Alternative Implementation 13, wherein each of the plurality of conformal sensors is an electromyography sensor.

Alternative Implementation 16. The system of Alternative Implementation 13, wherein one or more of the plurality of conformal sensors includes a hard-wired connection to the central processing unit such that at least some of the generated digital representations are received by the central processing unit via the hard-wired connection.

Alternative Implementation 17. The system of Alternative Implementation 13, wherein one or more of the plurality of conformal sensors are wirelessly connected to the central processing unit such that at least some of the generated digital representations are received by the central processing unit via the wireless connection.

Alternative Implementation 18. The system of Alternative Implementation 13, wherein the physiological profiles are stored in a library of physiological profiles stored in the at least one second memory, the at least one first memory, or both.

Alternative Implementation 19. The system of Alternative Implementation 13, wherein the physiological status of the subject indicates that the subject is walking, running, climbing, or crawling.

Alternative Implementation 20. The system of Alternative Implementation 13, wherein the physiological status of the subject indicates that the subject is exhausted, injured, has a dangerously high heart rate, has a dangerously high core body temperature, performing as expected, performing a specific function, or any combination thereof.

Alternative Implementation 21. The system of Alternative Implementation 13, wherein the instructions executable by the central processing unit further cause the central processing unit to transmit a signal from the central processing unit to mechanical components of utility gear worn by the subject in response to the comparison, the signal activating the utility gear to aid activity of the subject.

Alternative Implementation 22. The system of Alternative Implementation 21, wherein the mechanical components include an exoskeleton and the signal activate the exoskeleton to aid the subject's leg movement.

Alternative Implementation 23. The system of Alternative Implementation 13, wherein the physiological status is transmitted wirelessly by the central processing unit for receipt at a remote location.

Alternative Implementation 24. The system of Alternative Implementation 13, wherein one or more of the plurality of conformal sensors are integral with a layer of stretchable fabric material worn by the subject such that the conformal sensors are positioned adjacent to the outer skin surface of the subject.

Alternative Implementation 25. A system for monitoring physiological performance of a subject, the system comprising: a physiological conformal sensor configured to conform to a portion of an outer skin surface of the subject and to create digital signals representative of physiological data sensed by the physiological sensor; and a central controller coupled to the physiological conformal sensor, the central controller being configured to: (i) receive the digital signals from the physiological conformal sensor; (ii) determine a physiological stress index based on the received digital signals; and (iii) analyze the determined physiological stress index to determine if the subject is at risk or not at risk of reaching dangerous levels of stress.

Alternative Implementation 26. The system of Alternative Implementation 25, wherein in response to an at risk determination being made by the central controller, the central controller is caused to send an alert to the subject, to a third party, or both.

Alternative Implementation 27. The system of Alternative Implementation 25, wherein the physiological conformal sensor includes a heart rate sensor for sensing a heart rate of the subject and a core body temperature sensor for estimating a core body temperature of the subject.

Alternative Implementation 28. The system of Alternative Implementation 27, wherein at least a portion of the received digital signals is representative of the heart rate and the core body temperature of the subject.

Alternative Implementation 29. The system of Alternative Implementation 28, wherein the determined physiological stress index condition is transmitted wirelessly by the central controller to the third party.

Alternative Implementation 30. A system comprising: a plurality of conformal sensors, each conformal sensor including a processing portion and an electrode portion, the electrode portion being configured to substantially conform to a portion of an outer skin surface of a subject and to sense a parameter of the subject, the electrode portion generating a parameter signal which is transmitted from the electrode portion to the processing portion, the processing portion being configured to create processed signals based on the parameter signal; and a central controller coupled to each of the plurality of conformal sensors and being configured to receive the processed signals from each of the plurality of conformal sensors.

Alternative Implementation 31. A system comprising: a plurality of conformal sensors, at least a portion of each of the conformal sensors being configured to substantially conform to a portion of an outer skin surface of a subject and to sense a parameter of the subject and generate a parameter signal based on the sensed parameter; and a central controller coupled to each of the plurality of conformal sensors and being configured to receive the parameter signals from each of the plurality of conformal sensors.

It is contemplated that any element or elements from any one of the above implementations (e.g., implementations 1-31) can be combined with any other element or elements from any of the other ones of the above implementations (e.g., implementations 1-31) to provide one or more additional alternative implementations.

Each of the above concepts and obvious variations thereof is contemplated as falling within the spirit and scope of the claimed invention, which is set forth in the following claims.

1. A system for calculating a physiological stress index of a subject, the system comprising:
a plurality of conformal, stretchable, and flexible sensors, each conformal, stretchable, and flexible sensor including a conformal and flexible substrate with a processing portion, an accelerometer, and an electrode portion coupled thereto, the electrode portion of a first group of the plurality of conformal sensors being configured to substantially conform to a first portion of an outer skin surface of the subject adjacent to a first muscle of the subject and to sense electrical pulses generated by the first muscle and the electrode portion of a second group of the plurality of conformal sensors, that is distinct from the first group, being configured to substantially conform to a second portion of the outer skin surface of the subject adjacent to a second muscle of the subject and to sense electrical pulses generated by the second muscle, the accelerometer being configured to sense motion, a first one of the plurality of conformal, stretchable, and flexible sensors including (i) a heart rate sensor for sensing a heart rate of the subject and (ii) a temperature sensor for sensing a core body temperature of the subject, the heart rate sensor and temperature sensor being coupled to the conformal and flexible substrate of the first conformal, stretchable, and flexible sensor, for each of the plurality of conformal, stretchable, and flexible sensors, the sensed electrical pulses and the sensed motion are transmitted to the processing portion as raw analog signals for onboard processing thereof by the processing portion of the conformal, stretchable, and flexible sensor, for the first conformal, stretchable, and flexible sensor, the sensed heart rate and the sensed core body temperature are transmitted from the heart rate sensor and the temperature sensor to the processing portion of the first conformal, stretchable, and flexible sensor as raw analog signals for onboard processing thereof, for each of the plurality of conformal, stretchable, and flexible sensors, the processing portion is configured to create digital signals representative of the raw analog signals; and
a central controller coupled to each of the plurality of conformal, stretchable, and flexible sensors and being configured to receive the digital signals from each of the plurality of conformal sensors, the central controller further being configured to (i) determine a physiological stress index as a function of an initial heart rate (HR₍₀₎), a subsequent heart rate (HR₍ₜ₎), an initial core body temperature (T_{core(0)}), and a subsequent core body temperature (T_{core(t)}) and (ii) based on the determined physiological stress index, cause an alert to be transmitted.

2-5. (canceled)

6. The system of claim 1, wherein the plurality of conformal, stretchable, and flexible sensors are electromyography sensors.

7. The system of claim 1, wherein one or more of the plurality of conformal, stretchable, and flexible sensors includes a hard-wired connection to the central controller such that at least some of the raw analog signals are received by the central controller via the hard-wired connection.

8. The system of claim 1, wherein one or more of the plurality of conformal, stretchable, and flexible sensors are wirelessly connected to the central controller such that at least some of the raw analog signals are received by the central controller via the wireless connection.

9-34. (canceled)

35. The system of claim 1, wherein the alert is transmitted by the central controller to a hand-held device associated with the subject, a third party, or both, responsive to the determined physiological stress index exceeding a predefined level.

36-38. (canceled)

39. The system of claim 35, wherein the predefined level is greater than 7.5 out of 10.

40. The system of claim 1, wherein the function used by the central controller to determine the physiological stress index is 5*(T_{core(t)}−T_{core(0)})*(39.5−T_{core(0)})⁻¹+5*(HR₍ₜ₎−HR₍₀₎)*(180−HR₍₀₎)⁻¹, where T_{core(t)} is the core body temperature in Celsius of the subject at time t, T_{core(0)} is the core body temperature in Celsius of the subject at time 0, HR₍ₜ₎ is the heart rate of the subject at time t, and HR₍₀₎ is the heart rate of the subject at time 0.

41. The system of claim 1, wherein each of the plurality of conformal, stretchable, and flexible sensors has a thickness between about 500 micrometers and about 5 micrometers.

42. The system of claim 1, further comprising a chest wrap coupled with the first conformal, stretchable, and flexible sensor such that donning of the chest wrap about a chest of the subject automatically positions the first conformal, stretchable, and flexible sensor at a desired location on the chest of the subject.

43. A system for calculating a physiological stress index of a mammal, the system comprising:
a conformal, stretchable, and flexible sensor, the conformal, stretchable, and flexible sensor including (i) a conformal and flexible substrate, (ii) a heart rate sensor for sensing a heart rate of the mammal, (iii) a temperature sensor for sensing a core body temperature of the mammal, and (iv) a processing portion, the heart rate sensor and temperature sensor being coupled to the conformal and flexible substrate, the sensed heart rate and the sensed core body temperature being transmitted from the heart rate sensor and the temperature sensor to the processing portion as raw analog signals for onboard processing thereof, the processing portion being configured to create digital signals representative of the raw analog signals; and
a central controller coupled to the conformal, stretchable, and flexible sensor and being configured to (i) receive the digital signals from the conformal, stretchable, and flexible sensor, (ii) determine a physiological stress index as a function of an initial heart rate (HR₍₀₎), a subsequent heart rate (HR₍ₜ₎), an initial core body temperature (T_{core(0)}), and a subsequent core body temperature (T_{core(t)} and (iii) based on the determined physiological stress index, cause an alert to be transmitted.

44. The system of claim 43, wherein the conformal, stretchable, and flexible sensor includes a hard-wired connection to the central controller such that the raw analog signals are received by the central controller via the hard-wired connection.

45. The system of claim 43, wherein the conformal, stretchable, and flexible sensor is wirelessly connected to the central controller such that the raw analog signals are received by the central controller via the wireless connection.

46. The system of claim 43, wherein the alert is transmitted by the central controller to a hand-held device associated with the mammal, a third party, or both, responsive to the determined physiological stress index exceeding a predefined level.

47. The system of claim 46, wherein the predefined level is greater than 7.5 out of 10.

48. The system of claim 43, wherein the function used by the central controller to determine the physiological stress index is 5*(T_{core(t)}−T_{core(0)})*(39.5−T_{core(0)})⁻¹+5*(HR₍ₜ₎−HR₍₀₎)*(180−HR₍₀₎)⁻¹, where T_{core(t)} is the core temperature in Celsius of the subject at time t, T_{core(0)} is the core temperature in Celsius of the subject at time 0, HR₍ₜ₎ is the heart rate of the subject at time t, and HR₍₀₎ is the heart rate of the subject at time 0.

49. The system of claim 41, wherein the conformal, stretchable, and flexible sensor has a thickness between about 500 micrometers and about 5 micrometers.

50. The system of claim 41, further comprising a chest wrap coupled with the conformal, stretchable, and flexible sensor such that donning of the chest wrap about a chest of the mammal automatically positions the conformal, stretchable, and flexible sensor at a desired location on the chest of the mammal.

51. A system for monitoring physiological performance of a subject, the system comprising:
a physiological conformal sensor configured to conform to a portion of an outer skin surface of the subject and to create digital signals representative of physiological data sensed by the physiological sensor, the physiological conformal sensor having a thickness between about 500 micrometers and about 5 micrometers; and
a central controller coupled to the physiological conformal sensor, the central controller being configured to:
(i) receive the digital signals from the physiological conformal sensor;
(ii) determine a physiological stress index based on the received digital signals and an algorithm where the physiological stress index equals 5*(T_{core(t)}−T_{core(0)})*(39.5−T_{core(0)})⁻¹+5*(HR₍ₜ₎−HR₍₀₎)*(180−HR₍₀₎)⁻¹, where T_{core(t)} is the core temperature in Celsius of the subject at time t, T_{core(0)} is the core temperature in Celsius of the subject at time 0, HR₍ₜ₎ is the heart rate of the subject at time t, and HR₍₀₎ is the heart rate of the subject at time 0; and
(iii) analyze the determined physiological stress index to determine if the subject is at risk or not at risk of reaching dangerous levels of stress.

52. The system of claim 51, wherein in response to an at risk determination being made by the central controller, the central controller is caused to send an alert to the subject, to a third party, or both.

53. The system of claim 51, wherein the physiological conformal sensor includes a heart rate sensor for sensing a heart rate of the subject and a core body temperature sensor for estimating a core body temperature of the subject.

54. The system of claim 53, wherein at least a portion of the received digital signals is representative of the heart rate and the core body temperature of the subject.

55. The system of claim 54, wherein the determined physiological stress index condition is transmitted wirelessly by the central controller to the third party.

56. The system of claim 52, wherein the alert is sent by the central controller to a hand-held device associated with the subject, a third party, or both, responsive to the determined physiological stress index exceeding a predefined level.

57. The system of claim 56, wherein the predefined level is greater than 7.5 out of 10.

58. The system of claim 51, further comprising a chest wrap coupled with the physiological conformal sensor such that donning of the chest wrap about a chest of the subject automatically positions the physiological conformal sensor at a desired location on the chest of the subject.
